(19) United States
(12) Patent Application Publication
(10) Pub. No.: US 2018/0289247 A1
(43) Pub. Date: Oct. 11, 2018

(54) ENDOSCOPE SYSTEM

(71) Applicant: OLYMPUS CORPORATION, Tokyo (JP)

(72) Inventor: Soichiro KOSHIKA, Tokyo (JP)

(73) Assignee: OLYMPUS CORPORATION, Tokyo (JP)

(21) Appl. No.: 16/005,767

(22) Filed: Jun. 12, 2018

Related U.S. Application Data
(63) Continuation of application No. PCT/JP2016/076195, filed on Sep. 6, 2016.

(30) Foreign Application Priority Data
Dec. 14, 2015 (JP) 2015-243284

(51) Int. Cl. A61B 1/06; A61B 1/00; H04N 5/225

(52) U.S. Cl. CPC A61B 1/0661; A61B 1/0638; A61B 1/05; H04N 5/2256; A61B 1/00165

(57) ABSTRACT

An endoscope system includes: a light source that generates red, green, and blue laser lights; a light-guiding section including a first end portion into which the laser lights enter, and a second end portion from which the laser lights are applied to the subject; a light detection that detects reflection light from the subject, and outputs a detection signal according to the reflection light; a memory that stores a plurality of color correction parameters, each of which is set for each subject; and an image processing section that generates an observation image based on the detection signal, and performs color correction on the observation image based on at least one of the plurality of color correction parameters, which is selected according to the subject.

### CROSS REFERENCE TO RELATED APPLICATION

This application is a continuation application of PCT/JP2016/076195 filed on Sep. 6, 2016 and claims benefit of Japanese Application No. 2015-243284 filed in Japan on Dec. 14, 2015, the entire contents of which are incorporated herein by this reference.

### BACKGROUND OF INVENTION

### 1. Field of the Invention

The present invention relates to an endoscope system.

### 2. Description of the Related Art

Conventionally, a scanning endoscope apparatus, which is an endoscope apparatus used in medical fields, has been known, and such a scanning endoscope apparatus is configured to scan a subject with laser light which is narrow-band light having an excellent straight advancing ability and pick up an image of a subject. The scanning endoscope apparatus irradiates a subject with laser light while causing a distal end of an illumination optical fiber to oscillate, receives reflection light from the subject with a light-receiving optical fiber, and picks up an image of the subject. In the scanning endoscope apparatus, it is not necessary to provide a solid-state image pickup device in the insertion portion, which leads to a size reduction of the diameter of the insertion portion. Such a size reduction contributes to alleviation of a burden on the subject into which the insertion portion is inserted.

In addition, as another prior art example, Japanese Patent Application Laid-Open Publication No. 2008-302075 proposes the endoscope apparatus which irradiates a subject with illumination light from a lamp to pick up an image of the subject, and performs color conversion processing on an observation image in accordance with a scope, to thereby improve the accuracy of color reproduction in the observation image.

The color of the subject detected by the endoscope apparatus is different depending on the components of the light applied to the subject and the spectral reflection characteristic of the subject. It is known that the spectral reflection characteristic differs depending on the material and the like of the subject.

### SUMMARY OF THE INVENTION

An endoscope system according to one aspect of the present invention includes: a light source that generates red, green, and blue laser lights; a light-guiding section including a first end portion into which the laser lights enter, and a second end portion from which the laser lights are applied to the subject; a light detection that detects reflection light from the subject, and outputs a detection signal according to the reflection light; a memory that stores a plurality of color correction parameters, each of which is set for each subject; and an image processing section that generates an observation image based on the detection signal, and performs color correction on the observation image based on at least one of the plurality of color correction parameters, which is selected according to the subject.

### BRIEF DESCRIPTION OF THE DRAWINGS

FIG. 1 is a block diagram illustrating a configuration of a main part of an endoscope system according to an embodiment of the present invention.

FIG. 2 is a sectional view illustrating a configuration of an actuator of the endoscope system according to the embodiment of the present invention.

FIG. 3 is an explanatory view describing a spiral-shaped scanning path of the endoscope system according to the embodiment of the present invention.

FIG. 4 is an explanatory view describing the spiral-shaped scanning path of the endoscope system according to the embodiment of the present invention.

FIG. 5 is a block diagram illustrating a configuration of an image processing section of the endoscope system according to the embodiment of the present invention.

FIG. 6 is an explanatory view describing a reference red reflection light intensity of a color chart according to the embodiment of the present invention.

FIG. 7 is an explanatory view describing a reference green reflection light intensity of the color chart according to the embodiment of the present invention.

FIG. 8 is an explanatory view describing a reference blue reflection light intensity of the color chart according to the embodiment of the present invention.

FIG. 9 is an explanatory view describing red, green, and blue reflection light intensities of the color chart according to the embodiment of the present invention.

FIG. 10 is a flowchart showing an example of a color correction parameter setting flow in the endoscope system according to the present embodiment of the present invention.

FIG. 11 illustrates a spectral reflection characteristic of a nasal mucosa in the endoscope system according to the embodiment of the present invention.

FIG. 12 illustrates a spectral reflection characteristic of nasal drip in the endoscope system according to the embodiment of the present invention.

FIG. 13 is an explanatory view describing an image processing of magnification chromatic aberration.

FIG. 14 is an explanatory view describing the image processing of magnification chromatic aberration.

FIG. 15 is an explanatory view describing the image processing of magnification chromatic aberration.

### DETAILED DESCRIPTION OF THE PREFERRED EMBODIMENT(S)

Hereinafter, an embodiment of the present invention will be described referring to drawings.

### (Configuration)

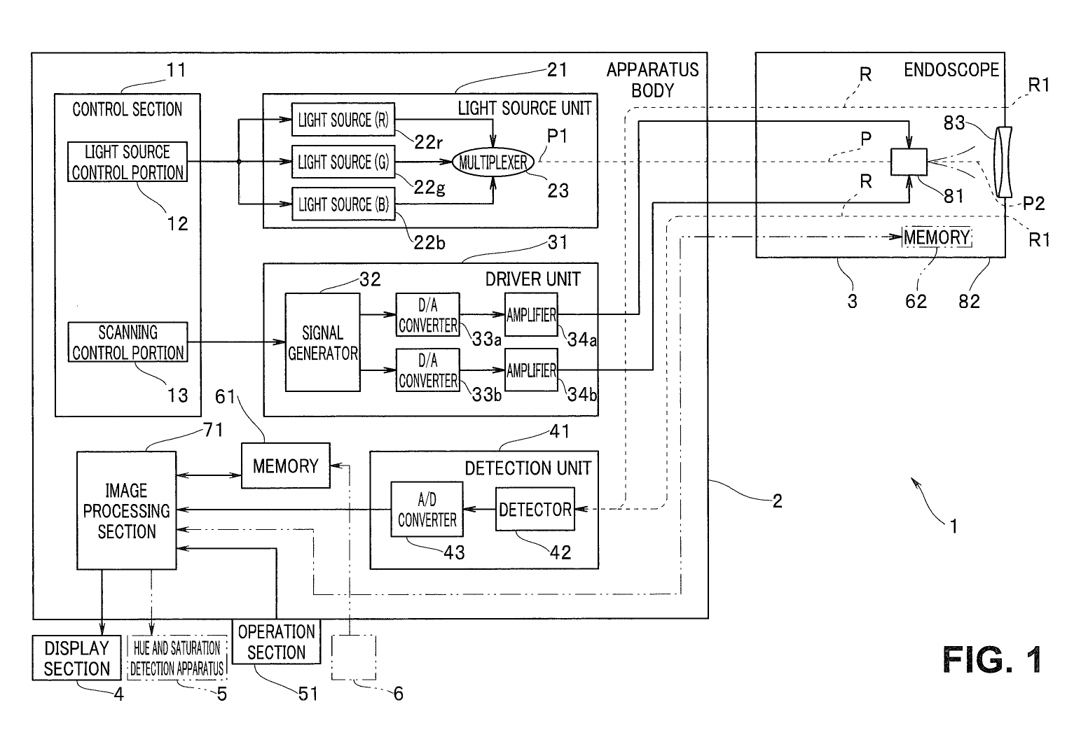

FIG. 1 is a block diagram illustrating a configuration of a main part of an endoscope system 1 according to an embodiment of the present invention. FIG. 1 illustrates electric signal lines with solid lines and optical fibers with dashed lines.

The main part of the endoscope system 1 includes an apparatus body 2, an endoscope 3, and a display section 4, as shown in FIG. 1. The apparatus body 2 is connected to the endoscope 3 and the display section 4.

The apparatus body 2 includes a control section 11, a light source unit 21 as a light source, a driver unit 31, a detection unit 41 as a light detection, an operation section 51, a memory 61, and an image processing section 71.

The control section 11 is a circuit that performs driving control of the light source unit 21 and the driver unit 31. The control section 11 includes a light source control portion 12 and a scanning control portion 13.

The light source control portion 12 is connected to the light source unit 21, and configured to output a control signal to the light source unit 21, to thereby be capable of performing driving control of the light source unit 21.

The scanning control portion 13 is connected to the driver unit 31, and configured to output a control signal to the driver unit 31, to thereby be capable of performing driving control of the driver unit 31.

The light source unit 21 is configured to generate red, green, and blue laser lights based on the control signals inputted from the light source control portion 12 and enable the laser lights to enter an incident side end portion P1 of an illumination optical fiber P which is a light-guiding section. For example, the light source unit 21 sequentially and repeatedly generates the red, green, and blue laser lights based on the control signals inputted from the light source control portion 12, to cause the laser lights to enter the incident side end portion P1 of the illumination optical fiber P.

The light source unit 21 includes red, green, and blue laser light sources 22r, 22g, and 22b, and a multiplexer 23. The light source unit 21 is connected to the illumination optical fiber P.

The respective red, green, and blue laser light sources 22r, 22g, and 22b are connected to the multiplexer 23.

The red laser light source 22r generates red laser light. The green laser light source 22g generates green laser light. The blue laser light source 22b generates blue laser light.

The multiplexer 23 is configured to be capable of multiplexing wavelengths of the lights inputted from the respective red, green, and blue laser light sources 22r, 22g, and 22b. The multiplexer 23 is connected to the incident side end portion P1 of the illumination optical fiber P. The multiplexer 23 multiplexes the wavelengths of the inputted lights and outputs the wavelength-multiplexed light to the illumination optical fiber P.

The illumination optical fiber P includes the incident side end portion P1 which is a first end portion into which light enters, and irradiation side end portion P2 which is a second end portion from which light is applied to the subject. The illumination optical fiber P is configured to be capable of guiding the light from the incident side end portion P1 to the irradiation side end portion P2. The illumination optical fiber P allows the light inputted from the multiplexer 23 to be applied from a distal end of an insertion portion 82 of the endoscope 3 to a subject.

The driver unit 31 is a circuit that drives an actuator 81 of the endoscope 3, to cause the irradiation side end portion P2 of the illumination optical fiber P to oscillate. The driver unit 31 includes a signal generator 32, D/A converters 33a, 33b, and amplifiers 34a, 34b. The one-dot-chain lines in FIG. 1 schematically illustrate a state in which the irradiation side end portion P2 oscillates.

The signal generator 32 generates drive signals AX and AY for driving the actuator 81 based on the control signals inputted from the scanning control portion 13, and outputs the generated drive signals to the D/A converters 33a, 33b.

The drive signal AX is outputted so as to enable the irradiation side end portion P2 of the illumination optical fiber P to oscillate in an X-axis direction. The drive signal AX is defined by the following expression (1), for example. In the following expression (1), X(t) represents the signal level of the drive signal AX at a time t, Amx represents an amplitude value which is independent of the time t, and G(t) represents a predetermined function that modulates a sine wave sin(2πft).

X(t)=Amx×G(t)×sin(2πft) (1)

The drive signal AY is outputted so as to enable the irradiation side end portion P2 of illumination optical fiber P to oscillate in a Y-axis direction. The drive signal AY is defined by the following expression (2), for example. In the following expression (2), Y(t) represents the signal level of the drive signal AY at the time t, Amy represents an amplitude value which is independent of the time t, G(t) represents a predetermined function that modulates a sine wave sin(2πft+φ), and φ represents a phase.

Y(t)=Amy×G(t)×sin(2πft+φ) (2)

The D/A converters 33a, 33b convert the drive signals AX, AY inputted from the signal generator 32 from digital signals respectively to analog signals, and output the analog signals to the amplifiers 34a and 34b.

The amplifiers 34a and 34b amplify the drive signals AX, AY inputted from the D/A converters 33a and 33b, and output the amplified drive signals AX, AY to the actuator 81.

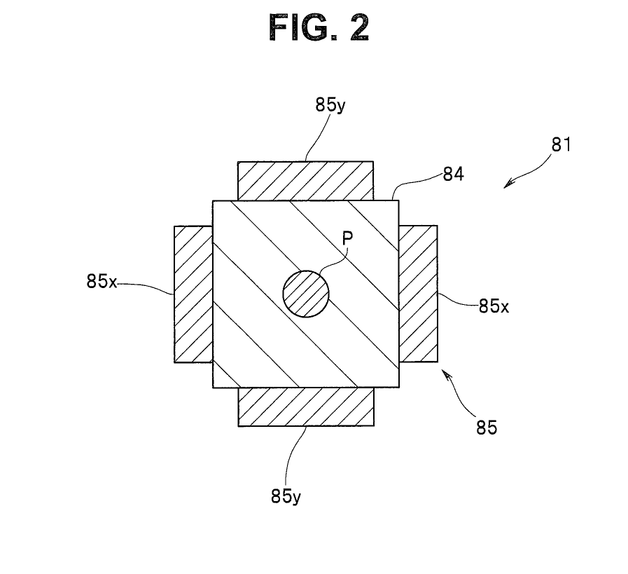

FIG. 2 is a sectional view illustrating the configuration of the actuator 81 of endoscope system 1 according to the embodiment of the present invention.

The endoscope 3 is inserted into a subject, and configured to be capable of irradiating the subject with the light emitted from the light source unit 21, to pick up an image of the reflection light from the subject. The endoscope 3 includes the insertion portion 82, the actuator 81, a lens 83, and a light-receiving portion R1.

The insertion portion 82 is formed in an elongated shape, and configured to be insertable into the subject.

The actuator 81 is capable of causing the irradiation side end portion P2 to oscillate, and moving the light application position of the laser lights along a predetermined scanning path. The predetermined scanning path is a spiral-shaped scanning path, for example. As shown in FIG. 2, the actuator 81 includes a ferrule 84 and piezoelectric elements 85.

The ferrule 84 is made of zirconia (ceramic), for example. The ferrule 84 is provided in the vicinity of the irradiation side end portion P2 such that the irradiation side end portion P2 of the illumination optical fiber P can oscillate.

Each of the piezoelectric elements 85 has a polarization direction which is individually set in advance, and vibrates in response to the drive signals AX, AY inputted from the driver unit 31, to thereby be capable of causing the irradiation side end portion P2 of illumination optical fiber P to oscillate. The piezoelectric elements 85 include an X-axis piezoelectric element 85x for causing the illumination optical fiber P to oscillate in the X-axis direction orthogonal to the longitudinal axis of the illumination optical fiber P, and a y-axis piezoelectric element 85y for causing the illumination optical fiber P to oscillate in the Y-axis direction which is a direction orthogonal to the longitudinal axis of illumination optical fiber P and the X-axis direction.

The lens 83 is provided at the distal end of the insertion portion 82, and configured to be capable of receiving the light applied from the irradiation side end portion P2 of the illumination optical fiber P, and the light is applied to the subject through the lens 83.

The light-receiving portion R1 is provided at the distal end of the insertion portion 82, and receives the reflection light from the subject. The received reflection light from the subject is outputted to the detection unit 41 of the apparatus body 2 through the light-receiving optical fiber R.

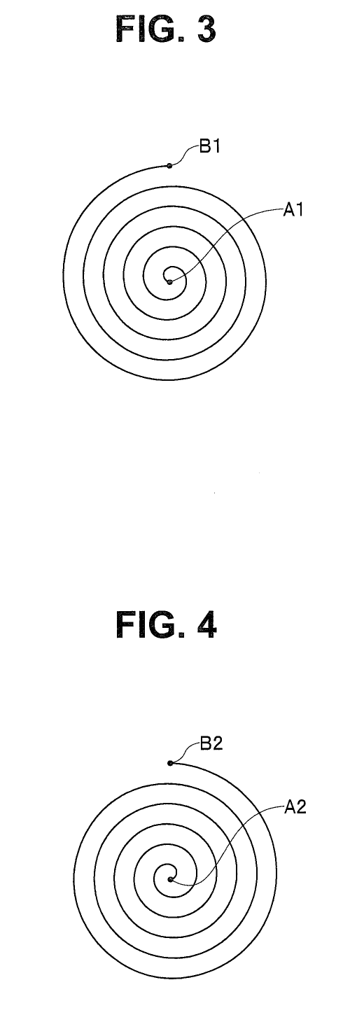

FIG. 3 and FIG. 4 are explanatory views describing the spiral-shaped scanning path of the endoscope system 1 according to the embodiment of the present invention.

When the driver unit 31 outputs the drive signals AX, AY, while increasing the signal levels of the drive signals, the illumination optical fiber P is oscillated by the actuator 81, and the light application position of the illumination optical fiber P moves along the spiral-shaped scanning path which gradually gets away from the center, as shown by A1 to B1 in FIG. 3. After that, when the driver unit 31 outputs the drive signals AX, AY, while decreasing the signal levels of the drive signals, the light application position of the illumination optical fiber P moves along the spiral-shaped scanning path which gradually gets close to the center, as shown by B2 to A2 in FIG. 4. As a result, the red, green, and blue laser lights sequentially generated by the light source unit 21 are applied spirally to the subject, the reflection light from the subject is received by the light-receiving portion R1, and the subject is spirally scanned.

Returning to FIG. 1, the detection unit 41 is a circuit that detects the reflection light from the subject and outputs a detection signal according to the detected reflection light to the image processing section 71. The detection unit 41 includes a detector 42 and an A/D converter 43.

The detector 42 includes, for example, a photoelectric conversion device such as an avalanche photodiode, and converts the reflection light from the subject, which is inputted from the light-receiving portion R1 through the light-receiving optical fiber R, into a detection signal, to output the detection signal to the A/D converter 43.

The A/D converter 43 converts the detection signal inputted from the detector 42 into a digital signal, to output the digital signal to the image processing section 71.

The operation section 51 includes a changeover switch to which an instruction for switching the observation mode is inputted by the operator. The operation section 51 is connected to the image processing section 71, and configured to be capable of outputting the instruction inputted by the operator to the image processing section 71. The operator inputs the instruction to the operation section 51, to thereby switch the observation mode and switch to the color correction parameter to be used for color correction among a plurality of color correction parameters.

A memory 61 is constituted of a rewritable nonvolatile memory. The plurality of color correction parameters set for the respective subjects are stored in the memory 61. The memory 61 is connected to the image processing section 71. The image processing section 71 is capable of referring to the color correction parameters stored in the memory 61.

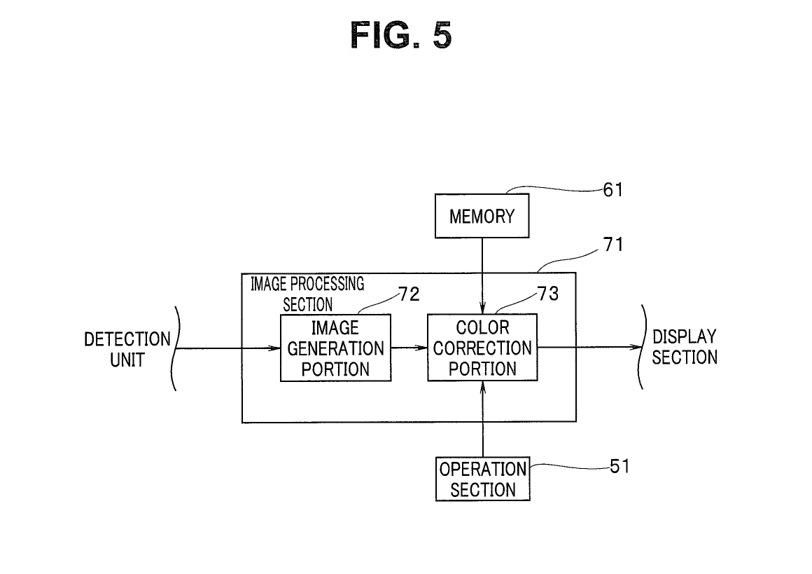

FIG. 5 is a block diagram illustrating the configuration of the image processing section 71 of the endoscope system 1 according to the embodiment of the present invention.

The image processing section 71 is a circuit that generates an observation image based on the detection signal inputted from the detection unit 41, to perform color correction on the observation image based on at least one color correction parameter, which is among the plurality of color correction parameters, selected according to the subject.

The image processing section 71 is a circuit including an image generation portion 72 and a color correction portion 73.

The image generation portion 72 receives the detection signal from the detection unit 41, converts the detection signal into image information referring to a mapping table, not shown, and generates an observation image frame by frame. The observation image generated by the image processing section 71 is outputted to the color correction portion 73. The observation image includes red, green, and blue signal values.

In order to generate a more preferable observation image, the image processing section 71 may use only the detection signal detected along either the spiral-shaped scanning path (from A1 to B1 in FIG. 3) which gradually gets away from the center or the spiral-shaped scanning path (from B2 to A2 in FIG. 4) which gradually gets close to the center, to generate an observation image.

The color correction portion 73 is configured to be capable of performing color correction on the observation image according to the subject, and outputting the observation image subjected to the color correction to the display section 4. The color correction portion 73 is connected to the operation section 51, the memory 61, and the display section 4. The color correction portion 73 is configured to be capable of detecting the observation mode, the instruction for which is inputted from the operation section 51, acquiring the color correction parameter according to the observation mode from the memory 61, performing color correction on the observation image inputted from the image generation portion 72 by using the acquired color correction parameter, and outputting the observation image subjected to the color correction to the display section 4.

When the observation mode is switched to a nasal mucosa observation mode by the instruction inputted to the operation section 51, for example, the color correction portion 73 acquires the color correction parameter for nasal mucosa from the memory 61, corrects the color of the observation image using the color correction parameter for nasal mucosa, and outputs the observation image subjected to the color correction to the display section 4.

When the observation mode is switched to a nasal drip observation mode by the instruction inputted to the operation section 51, for example, the color correction portion 73 acquires the color correction parameter for nasal drip from the memory 61, corrects the color of the observation image using the color correction parameter for nasal drip, to output the observation image subjected to the color correction to the display section 4.

The display section 4 is constituted of a monitor and the like, and is capable of displaying the observation image outputted from the image processing section 71, and the observation mode for indicating the subject as an observation target to the operator.

Next, description will be made on the color correction parameters.

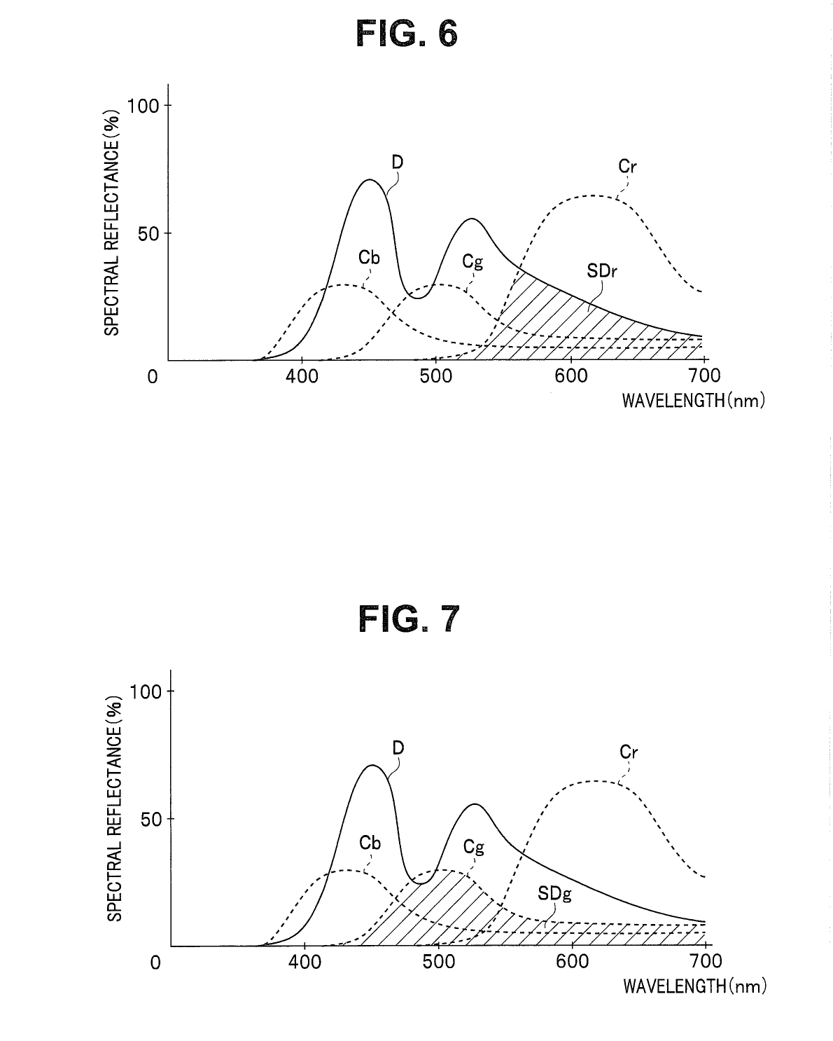

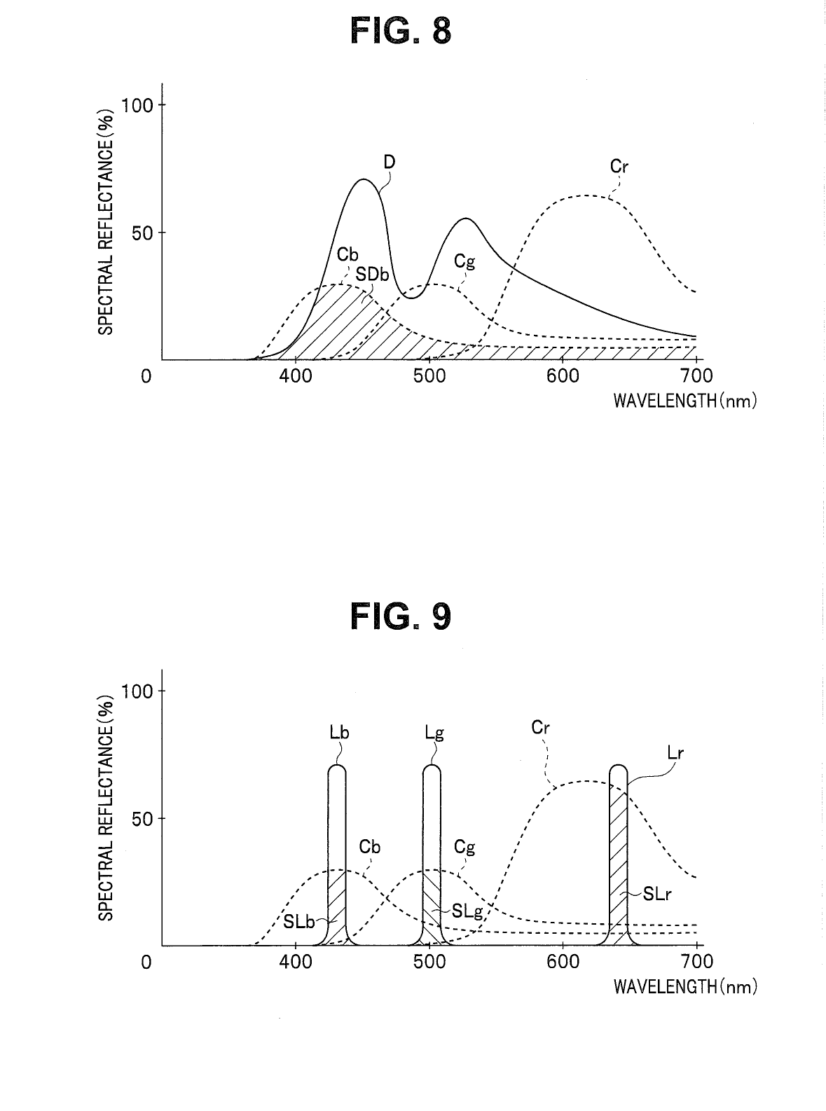

FIGS. 6, 7, and 8 are explanatory views for describing reference red, green, and blue reflection light intensities of the color chart according to the embodiment of the present invention. FIGS. 6, 7, and 8 illustrate the reflection light intensities of the respective colors when the LED light is applied to the color chart as a color sample from the endoscope serving as a reference endoscope. In FIG. 6, a red reflection light intensity SDr of the color chart is shown by the area of the part in which the light emission characteristic D of the LED and a red spectral reflection characteristic Cr of the color chart overlap with each other. In FIG. 7, a green reflection light intensity SDg of the color chart is shown by the area of the part in which the light emission characteristic D of the LED and a green spectral reflection characteristic Cg of the color chart overlap with each other. In FIG. 8, a blue reflection light intensity SDb of the color chart is shown by the area of the part in which the light emission characteristic D of the LED and a blue spectral reflection characteristic Cb of the color chart overlap with each other.

FIG. 9 is an explanatory view describing the red, green, and blue reflection light intensities of the color chart, according to the embodiment of the present invention. FIG. 9 illustrates the reflection light intensities when the respective laser lights from the endoscope 3 are applied to the color chart. The red reflection light intensity SLr of the color chart is shown by the area of the part in which the light emission characteristic Lr of the red laser light and the red spectral reflection characteristic Cr of the color chart overlap with each other. The green reflection light intensity SLg of the color chart is shown by the area of the part in which the light emission characteristic Lg of the green laser light and the green spectral reflection characteristic Cg of the color chart overlap with each other. The blue reflection light intensity SLb of the color chart is shown by the area of the part in which the light emission characteristic Lb of the blue laser light and the blue spectral reflection characteristic Cb of the color chart overlap with each other. In FIG. 9, the light emission characteristics Lr, Lg, and Lb of the laser lights are schematically illustrated such that the regions indicating the respective characteristics are shown wider than they really are, for the description purpose.

The color correction parameter is set in advance so that the hue and the saturation of an observation image can be corrected. The hue and the saturation of the observation image are determined depending on the ratio of the red, green, and blue signal values. When the green signal value is set as a reference, the red and blue signal values other than the green signal value are corrected, to thereby enable the hue and the saturation of the observation image to be corrected. Therefore, the color correction parameter includes a color correction parameter Yr by which the red signal value is multiplied and a color correction parameter Yb by which the blue signal value is multiplied.

The color correction parameter is set in advance according to the characteristics of the endoscope 3 such that the hue and the saturation that change depending on the characteristic of the endoscope 3 can be corrected.

A plurality of color correction parameters are set, in advance, for the respective subjects, according to the spectral reflection characteristics of the subjects. For example, the color correction parameters include the color correction parameter for nasal mucosa set according to the spectral reflection characteristic of the nasal mucosa and a color correction parameter for nasal drip set according to the spectral reflection characteristic of the nasal drip. For example, the color correction parameter for nasal mucosa includes a parameter value for decreasing the blue signal value.

The hue angle and saturation value of the observation image change according to the ratio of the red, green, and blue reflection light intensities. As shown in the following expression (3), when the ratio of the red, green, and blue reflection light intensities at the time when the image of the subject (color chart in the present embodiment) is picked up by applying the red, green, and blue laser lights by the endoscope 3 is equal to the ratio of the reflection light intensities at the time when the image of the subject is picked up by applying the LED lights from an endoscope serving as a reference (hereinafter, referred to as “reference endoscope”), not shown, the hue angle and saturation value of the subject, the image of which is picked up with the endoscope 3, is the same as the hue angle (hereinafter, referred to as “reference hue angle”) and the saturation value (hereinafter, referred to as “reference saturation value”) of the subject, the image of which is picked up with the reference endoscope.

SDr:SDg:SDb=SLr:SLg:SLb (3)

Based on the expression (3), the color correction parameters Xr (red), Xg (green), and Xb (blue) are represented by expressions (4), (5), and (6) shown below.

Xr=SDr/SLr (4)

Xg=SDg/SLg (5)

Xb=SDb/SLb (6)

If the color correction parameter Xg (green) is set as a reference, the color correction parameters Yr (red) and Yb (blue) are represented by the following expressions (7) and (8).

Yr=Xr/Xg (7)

Yb=Xb/Xg (8)

The observation image generated by the image generation portion 72 includes the red, green, and blue signal values corresponding to the reflection light intensities SLr, SLg, and SLb. The color correction portion 73 multiplies the red signal value by the color correction parameter Yr (red) and multiplies the blue signal value by the color correction parameter Yb (blue), with the green color as a reference, thereby capable of reproducing the colors of the color chart, the image of which is picked up by the reference endoscope, on the observation image inputted from the image generation portion 72.

Next, description will be made on the color correction parameter setting flow.

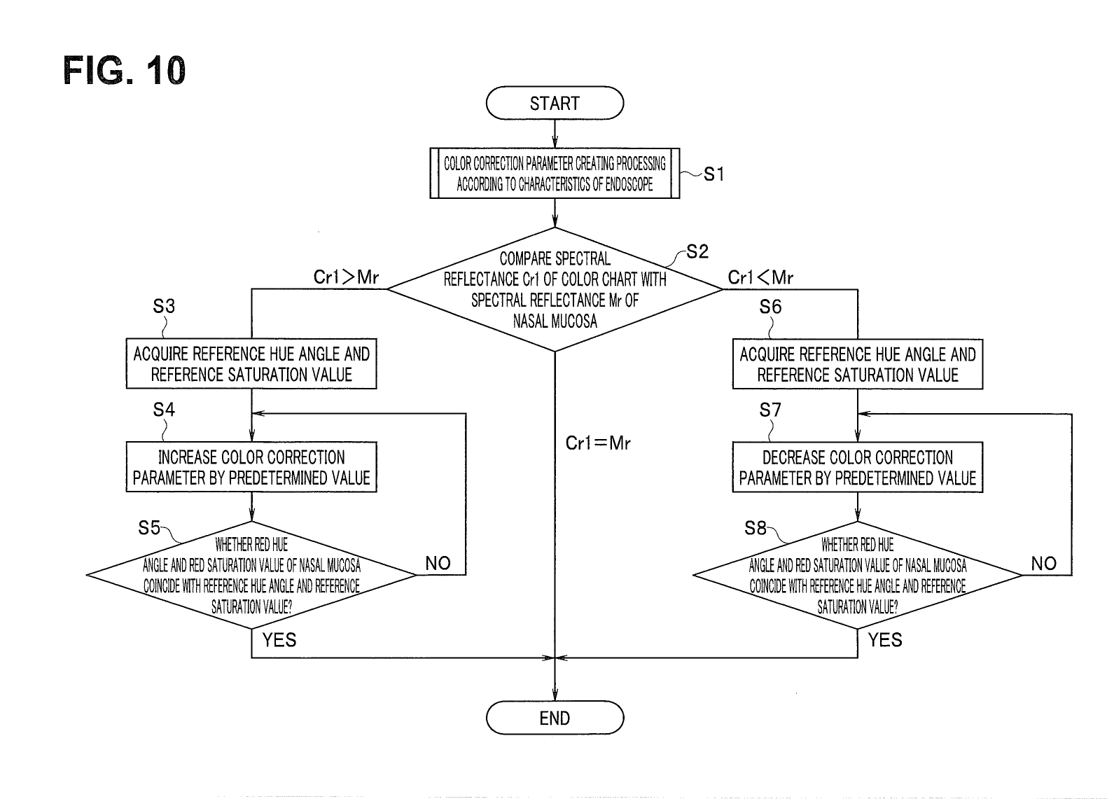

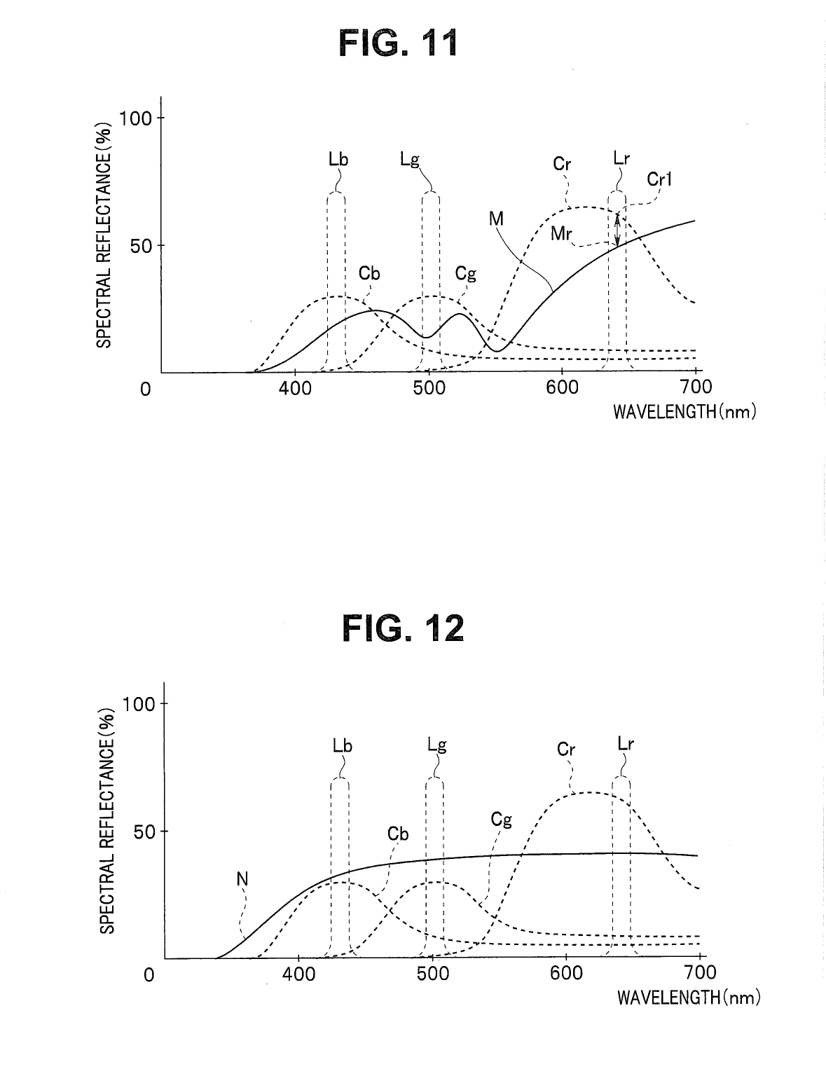

FIG. 10 is a flowchart showing an example of the color correction parameter setting flow in the endoscope system 1 according to the embodiment of the present invention. FIG. 10 exemplifies the setting flow of the red color correction parameter for the nasal mucosa. FIG. 11 illustrates a spectral reflection characteristic M of the nasal mucosa in the endoscope system 1 according to the embodiment of the present invention. FIG. 12 illustrates a spectral reflection characteristic N of the nasal drip in the endoscope system 1 according to the embodiment of the present invention.

The color correction parameters are set before the factory shipment of the endoscope system 1. The color correction parameters are set by the processing performed by a color correction parameter setting apparatus 6 (shown by the two-dot-chain line in FIG. 1) capable of executing a program by the CPU according to the flow shown in FIG. 10. Hereinafter, description will be made on the processing by the color correction parameter setting apparatus 6. Note that the color correction parameters may be set by manual operation according to the flow shown in FIG. 10.

The color correction parameter creating processing according to the characteristic of the endoscope 3 is performed (S1). In S1, the red reference hue angle and the red reference saturation value of the color chart are acquired. The reference hue angle and reference saturation value are set based on the detection result acquired by picking up, in advance, the image of the color chart by irradiating the color chart with the red LED light by the reference endoscope and outputted to a hue and saturation detection apparatus 5 such as a vector scope (shown by the two-dot-chain line in FIG. 1).

Next, the image of the color chart is picked up by irradiating the color chart with the red laser light by the endoscope 3, and the hue angle and the saturation value are outputted to the hue and saturation detection apparatus 5. The red color correction parameter Xr is corrected while being shifted by a predetermined value until the hue angle and the saturation value acquired by the endoscope 3 are brought into a state coincident with the reference hue angle and the reference saturation value, and then the red color correction parameter Xr is created. Note that the coincident state between the hue angle and the saturation value acquired by the endoscope 3 and the reference hue angle and the reference saturation value may include an error in an allowable range.

The spectral reflectance Crl of a color chart is compared with the spectral reflectance Mr of the nasal mucosa (S2). In S2, the spectral reflectance Crl of the color chart and the spectral reflectance Mr of the nasal mucosa in the wavelength of the laser light to be applied to the color chart are compared with each other, and when the spectral reflectance Crl of the color chart is larger than the spectral reflectance Mr of the nasal mucosa, the processing proceeds to S3. When the spectral reflectance Crl of the color chart is smaller than the spectral reflectance Mr of the nasal mucosa, the processing proceeds to S6. In addition, when the spectral reflectance Crl of the color chart is equal to the spectral reflectance Mr of the nasal mucosa, the processing is terminated. For example, in FIG. 11, in the wavelength of the red laser light Lr, since the spectral reflectance Crl of the color chart is larger than the spectral reflectance Mr of the nasal mucosa, the processing proceeds to S3.

In S3, the reference hue angle and the reference saturation value of the nasal mucosa are acquired. The reference hue angle and the reference saturation value of the nasal mucosa are set based on the detection result acquired by picking up, in advance, the image of the nasal mucosa by irradiating the nasal mucosa with the LED lights by the reference endoscope and outputted to the hue and saturation detection apparatus 5.

The color correction parameter Xr is increased by a predetermined value (S4). In S4, the color correction parameter Xr is increased by the predetermined value, the image of the nasal mucosa is picked up by irradiating the nasal mucosa with the red laser light by the endoscope 3, and an observation image is outputted.

Determination is made on whether the red hue angle and the red saturation value of the nasal mucosa in the observation image are in the state coincident with the reference hue angle and the reference saturation value of the nasal mucosa (S5). In S5, the hue angle and the saturation value of the observation image are outputted to the hue and saturation detection apparatus 5, to determine whether the hue angle and the saturation value of the observation image are in the state coincident with the reference hue angle and the reference saturation value acquired in S3. When not in the coincident state (S5: NO), the processing returns to S4. On the other hand, when in the coincident state, the processing is terminated.

In S6, the reference hue angle and the reference saturation value of the nasal mucosa are acquired by the reference endoscope.

The color correction parameter Xr is decreased by a predetermined value (S7). In S7, the color correction parameter Xr is decreased by the predetermined value, the image of the nasal mucosa is picked up by irradiating the nasal mucosa with the red laser light by the endoscope 3, and the observation image is outputted.

Determination is made on whether the red hue angle and the red saturation value of the nasal mucosa in the observation image are in the state coincident with the reference hue angle and the reference saturation value (S8). In S8, the hue angle and the saturation value of the observation image are outputted to the hue and saturation detection apparatus 5, and then determination is made on whether the hue angle and the saturation value of the observation image are in the state coincident with the reference hue angle and the reference saturation value acquired in S6. When not in the coincident state (S8: NO), the processing returns to S6. On the other hand, when in the coincident state, the processing is terminated.

The red color correction parameter Xr is set by performing the processing from S1 to S8.

The green color correction parameter Xg and the blue color correction parameter Xb are set by performing the processing same as that described above.

Calculation is performed with the expressions (7) and (8) based on the color correction parameters Xr, Xg, and Xb, and the color correction parameters Yr and Yb are set.

FIG. 12 illustrates the spectral reflection characteristic of the nasal drip. Also with regard to the nasal drip, the color correction parameters Xr, Xg, and Xb are set by performing the processing from S1 to S8, and the color correction parameters Yr and Yb are set with the expressions (7) and (8).

According to the embodiment, the image of the subject is picked up by irradiating the subject with the laser lights, and the color reproduction performance according to the subject can be improved.

### Modified Example 1 of the Embodiment

In the above-described embodiment, the observation mode is switched by inputting the instruction to the operation section 51. However, the observation mode may be switched based on the hue angle and the saturation value of the entire observation image.

In the modified example 1 of the embodiment, the image processing section 71 detects the proportion of a predetermined color in the observation image, and determines at least one color correction parameter among the plurality of color correction parameters according to the detected proportion of the predetermined color.

More specifically, the color correction portion 73 detects a feature region having the predetermined hue and saturation from the observation image inputted by the image generation portion 72. When the proportion of the detected feature region to the observation image is equal to or larger than a predetermined value, the color correction portion 73 switches the observation mode to a predetermined observation mode and determines the predetermined color correction parameter from among the plurality of color correction parameters stored in the memory 61.

For example, when the proportion of a red feature region to the observation image is equal to or larger than a predetermined value, the color correction portion 73 switches the observation mode to the nasal mucosa mode, to perform color correction by using the color correction parameter for nasal mucosa. In addition, when the proportion of a feature region characterized by yellow color to the observation image is equal to or larger than a predetermined value, the color correction portion 73 switches the observation mode to the nasal drip mode, to perform color correction by using the color correction parameter for nasal drip.

With the modified example 1 of the embodiment, switching of the color correction parameter is performed according to the color of the subject, the image of the subject is picked up by irradiating the subject with the laser lights, and the color reproduction performance according to the subject can be improved.

### Modified Example 2 of the Embodiment

In the modified example 1 of the embodiment, the observation mode is switched according to the hue and the saturation of the entire observation image. However, the observation mode may be switched for each of a plurality of small regions that constitute the observation image.

The color correction portion 73 detects a proportion of a predetermined color to each of the plurality of small regions that constitute the observation image, and determines at least one color correction parameter from among the plurality of color correction parameters depending on the detected proportion of the predetermined color.

The color correction portion 73 smoothes the color of the boundary between the small regions adjacent to each other. The color correction portion 73 performs smoothing processing on the pixels or the color correction parameters at the boundary of the small regions adjacent to each other, to shade off the colors of the small regions, to perform image processing for enabling natural transition of the colors.

With the modified example 2 of the embodiment, the color correction parameter is set for each of the small regions according to the color of the subject, the image of the subject is picked up by irradiating the subject with the laser lights, and the color reproduction performance according to the color of the subject can be improved.

Note that, in the above-described embodiment, the color correction portion 73 performs color correction by using the color correction parameter corresponding to one observation mode. However, the color correction may be performed by calculating the color correction parameter by performing calculation based on the color correction parameters corresponding to the plurality of observation modes.

The color correction parameter may be calculated as an intermediate correction parameter by calculating an average value of the color correction parameter for nasal mucosa and the color correction parameter for nasal drip, for example.

As another calculation example of the color correction parameter, for example, if attenuation of a light component of a particular color such as red occurs according to the length of the endoscope 3, the color correction parameter may be calculated by multiplying by a predetermined adjustment factor so as to enable the attenuation amount of light of a particular color to be complemented.

As another calculation example of the color correction parameter, for example, if the display section 4 includes a color correction function, the color correction parameter may be set in accordance with the characteristic of the color correction function of the display section 4.

Note that the memory 61 is provided in the apparatus body 2 in the above-described embodiment. However, a memory 62 may be provided in the endoscope 3.

In the above-described embodiment, one detector 42 is provided. However, three detectors for red, green, and blue colors may be respectively provided.

In the above-described embodiment, the color correction parameter for nasal mucosa and the color correction parameter for nasal drip are exemplified as the color correction parameter. However, the color correction parameter is not limited to the color correction parameter for nasal mucosa and the color correction parameter nasal drip. The color correction parameter may be the one used for image pickup of another subject.

### (Image Processing for Magnification Chromatic Aberration Correction)

The light reflected by the subject refracts when passing through the lens. However, the refractive index of light differs depending on the wavelength of the light, that is, the color of the light. As a result, the focal length of the lens differs depending on the color of the light, which causes a color shift at a peripheral edge portion of the lens. The color shift at the peripheral edge portion of the lens is corrected by the image processing for magnification chromatic aberration correction.

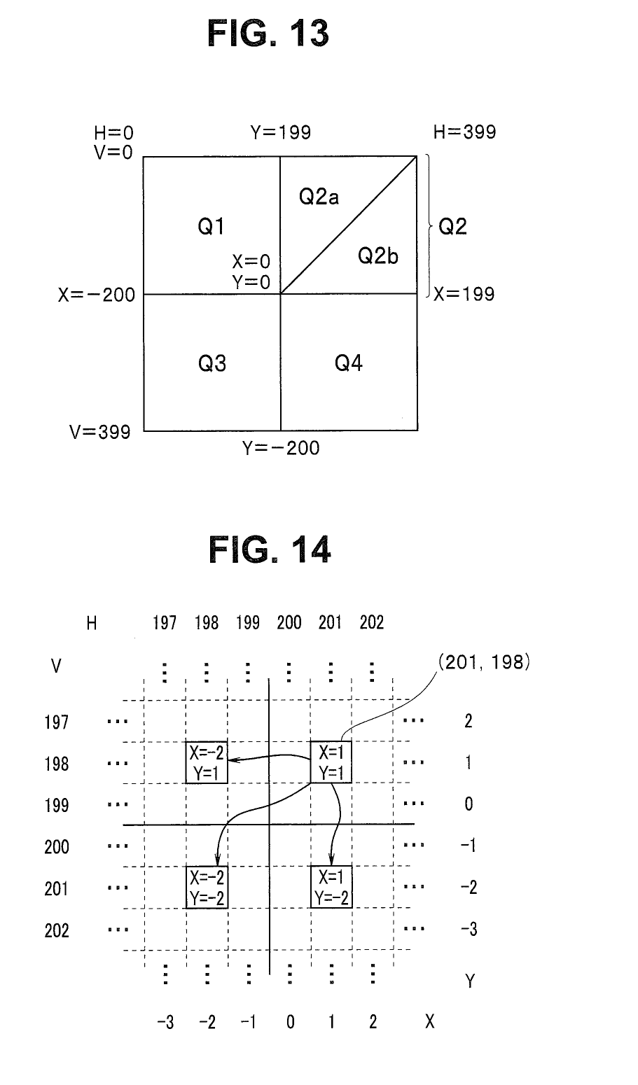

FIGS. 13, 14, and 15 are explanatory views for describing the image processing of the magnification chromatic aberration. The coordinates indicated by H and V in FIG. 13 show the coordinates in a television coordinate system, and the coordinates indicated by X and Y are coordinates in the center coordinate system.

A television coordinate system transformation table of a region Q2b which is ⅛ quadrant is stored in the memory. The television coordinate system transformation table includes reference source coordinate data Hn, Vn of pixels and reference destination coordinate data ΔHn, ΔVn of pixels, for each of all the pixels arranged in the region Q2b in the television coordinate system. The reference destination coordinate data ΔHn, ΔVn are defined as moving amounts of the pixels.

The image processing apparatus refers to the television coordinate system transformation table of the ⅛ quadrant, to thereby be capable of creating the center coordinate system transformation table of four quadrants. The image processing apparatus refers to the created center coordinate system transformation table, acquires coordinate data Xn, Yn of the pixels and reference destination coordinate data ΔXn, ΔYn, for each of all the pixels included in the center coordinate system transformation table, replaces the pixel values of the reference source coordinates (Xn, Yn) with the pixel values of the reference destination coordinates (Xn+Δx, Yn+ΔY), to thereby cause the pixels to move. As a result, magnification chromatic aberration can be corrected.

Creation of the center coordinate system transformation table is performed as follows.

The image processing apparatus performs vertical/horizontal inversion on the television coordinate system transformation table of the region Q2b, to create the television coordinate system transformation table of the region Q2a. Specifically, all the reference source coordinate data Hn and Vn included in the region Q2b are exchanged with each other and the reference destination coordinate data ΔHn and ΔVn are exchanged with each other, to thereby create the television coordinate system transformation table of the region Q2a.

The image processing apparatus creates the television coordinate system transformation table of the region Q2 by combining the television coordinate system transformation table of the region Q2a and the television coordinate system transformation table of the region Q2b.

The image processing apparatus creates the center coordinate system transformation table by performing calculation using the following expressions. The center coordinate system transformation table includes the reference source coordinate data Xn, Yn of the pixels and the reference destination coordinate data ΔXn, ΔYn of the pixels. The reference destination coordinate data ΔXn, ΔYn are defined as the moving amount of pixels.

For the region Q1,

Xn=199−Hn

Yn=199−Vn

ΔXn=−1×ΔHn

ΔYn=ΔVn

For the region Q2,

Xn=Hn−200

Yn=199−Vn

ΔXn=ΔHn

ΔYn=ΔVn

For the region Q3,

Xn=199−Hn

Yn=Vn−200

ΔXn=−1×ΔHn

ΔYn=−1×ΔVn

For the region Q4,

Xn=Hn−200

Yn=Vn−200

ΔXn=ΔHn

ΔYn=−1×ΔVn

For example, in FIG. 14, the coordinates (201, 198) in the television coordinate system is transformed into the coordinates (−2, 1) in the region Q1 in the center coordinate system, the coordinates (1, 1) in the region Q2 in the center coordinate system, the coordinates (−2, −2) in the region Q3 in the center coordinate system, and the coordinates (1, −2) in the region Q4 in the center coordinate system, and the center coordinate transformation table is created.

The image processing apparatus is capable of creating the transformation table of four quadrants based on the transformation table of ⅛ quadrant, which enables the storage quantity of the memory that stores the transformation table to be reduced.

In the magnification chromatic aberration correction, the image processing apparatus temporarily saves a copy of the observation image in the memory, acquires pixel values of the reference destination coordinates (Xn+ΔXn, Yn+ΔYn), referring to the observation image temporarily saved in the memory, to replace the pixel values of the reference source coordinates in the original observation image with the pixel values of the reference destination coordinates.

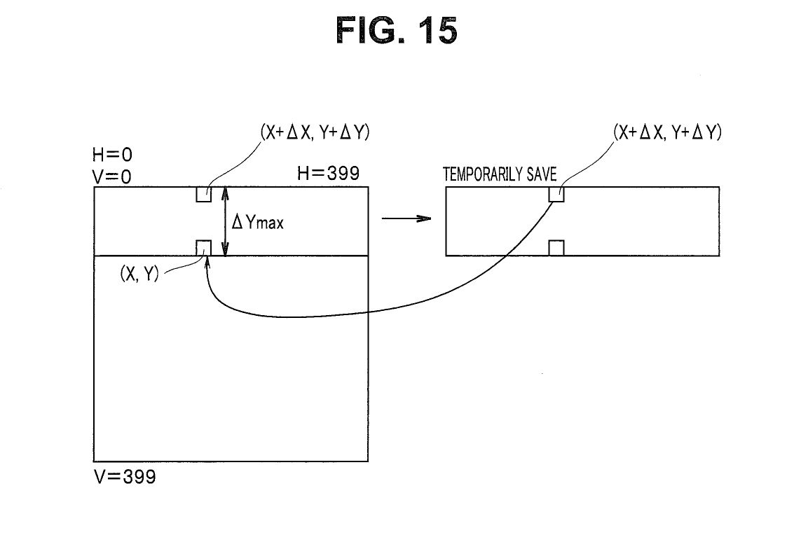

When temporarily saving the observation image, the image processing apparatus temporarily saves the observation image, in the memory, by the number of lines capable of covering the movement of the pixel that has the maximum moving amount. More specifically, the image processing apparatus acquires values of a plurality of reference destination coordinate data ΔYn from the respective pixels to be processed, and extracts the reference destination coordinate data ΔYmax indicating the maximum pixel moving amount, from the plurality of reference destination coordinate data ΔYn. Next, as shown in FIG. 15, the image processing apparatus extracts an image by the number of lines which is equal to the value of the reference destination coordinate data ΔYmax from the observation image, to temporarily save the extracted image in the memory.

The image processing apparatus is capable of reducing the storage quantity of the memory that temporarily saves the observation image.

The present invention is not limited to the above-described embodiment, and various changes, modifications, and the like are possible in a range without changing the gist of the present invention.

The present invention is capable of providing an endoscope system that picks up an image of a subject by irradiating the subject with laser lights and that is capable of improving the color reproduction performance according to the subject.

What is claimed is:

1. An endoscope system comprising:
a light source that generates red, green, and blue laser lights;
a light-guiding section including a first end portion into which the laser lights enter, and a second end portion from which the laser lights are applied to the subject;
a light detection that detects reflection light from the subject, and outputs a detection signal according to the reflection light;
a memory that stores a plurality of color correction parameters, each of which is set for each subject; and
an image processing section that generates an observation image based on the detection signal, and performs color correction on the observation image based on at least one of the plurality of color correction parameters, which is selected according to the subject.

2. The endoscope system according to claim 1, further comprising:
an actuator;
wherein the actuator causes the second end portion to oscillate, to thereby enable a light application position of the laser lights to move along a predetermined scanning path.

3. The endoscope system according to claim 2, wherein the actuator enables the light application position of the laser lights to shift along a spiral-shaped scanning path.

4. The endoscope system according to claim 1, wherein each of the plurality of color correction parameters is set for each subject according to a spectral reflection characteristic of the subject.

5. The endoscope system according to claim 1, wherein the plurality of color correction parameters include a color correction parameter for nasal mucosa set according to a spectral reflection characteristic of the nasal mucosa, and a color correction parameter for nasal drip set according to a spectral reflection characteristic of the nasal drip.

6. The endoscope system according to claim 5, wherein
the observation image includes red, green, and blue signal values, and
the color correction parameter for nasal mucosa includes a parameter value for decreasing the blue signal value.

7. The endoscope system according to claim 5, wherein the plurality of color correction parameters include an intermediate correction parameter calculated based on a first color correction parameter and a second color correction parameter.

8. The endoscope system according to claim 7, wherein
the first color correction parameter is the color correction parameter for nasal mucosa, and
the second color correction parameter is the color correction parameter for nasal drip.

9. The endoscope system according to claim 1, wherein the image processing section detects a proportion of a predetermined color in the observation image and determines at least one color correction parameter from among the plurality of color correction parameters according to the detected proportion of the predetermined color.

10. The endoscope system according to claim 1, wherein
the observation image is constituted of a plurality of small regions, and
the image processing section detects a proportion of a predetermined color for each of the plurality of small regions, and determines at least one color correction parameter from among the plurality of color correction parameters according to the detected proportion of the predetermined color, to perform color correction.

11. The endoscope system according to claim 1, wherein the image processing section smoothes a color of a boundary between small regions adjacent to each other.

12. The endoscope system according to claim 1, further comprising an operation section, wherein the color correction parameter can be switched to a color correction parameter to be used for color correction among the plurality of color correction parameters by an input of instruction to the operation section.
